(19) United States
(12) Patent Application Publication
(10) Pub. No.: US 2021/0237934 A1
(43) Pub. Date: Aug. 5, 2021

(54) Apparatus for smart utilization and storage of resources

(71) Applicant: Ramesh Kumburn, Fishers, IN (US)

(72) Inventor: Ramesh Kumburn, Fishers, IN (US)

(73) Assignees: Ramesh Kumburn, Fishers, IN (US); Moiz Neemuchwala, Fishers, IN (US); Sameeksha Kumburn, Fishers, IN (US); Ravindra K. Shetty, Bengaluru (IN)

(21) Appl. No.: 17/249,172

(22) Filed: Feb. 22, 2021

(51) Int. Cl. B65D 23/02; C02F 1/32; B65D 85/30; G06N 20/00; G16H 20/60; H02J 7/00

(52) U.S. Cl. CPC B65D 23/02; C02F 1/32; B65D 85/30; G06K 9/6256; G16H 20/60; H02J 7/007188; C02F 2201/3222; G06N 20/00

(57) ABSTRACT

A smart device to store resource being proposed. The resource may available in any state of matter. The smart device has built-in self-learning-based sensors and modules to measure, identify and purify the resources. Device has self-generating, built-in electricity generation and rechargeable battery modules. In one of the embodiments, the resource is available in liquid form, say water and the storage unit may be available in a transparent or opaque or colored unit. Smart sensors with chemical, Ion, bio feature sensing parameter are being embedded in the device with appropriate electronic modules. Resource is purified by various methods such as filtering, heating, Nano particle coating, smart magnets, additives of mineral enhancers, smart electrodes of different materials and electromagnetic wave and ultrasound treatments. Smart display would display various water quality parameters, such as temperature, oxygen and purity level, and diagnostic and prognostic information about resource and smart modules.

### BACKGROUND

These days several medical research study across the globe found that root cause of several diseases primarily depends on the consumption of quality of water. Therefore, everyone across the globe is looking for good quality drinking water, especially while traveling. Current portable water bottles available in the market only provide temperature information of the water in the portable bottle. There is a need for a smart device, which should able to identify the quality of water and purify it by various sensors and other methods. This invention demonstrates a self-learning sensor-based resource or water identifier and purifier product and solution. The proposed smart water bottle is a huge breakthrough for those of us trying to make healthy choices. This product would also track your water intake and synchronise with user's mobile health application, fitness devices and doctors either directly or through cloud environment in real time.

### SUMMARY

Embodiment of the inventions related to a modular design approach an apparatus for smart utilization and storage of resources being proposed.

In a further embodiment, the innovation is tailored to provide quality of healthy life by providing high quality pure water.

In a further embodiment, the novel technique been implemented for water bottle and it can be used for any type of liquid or any of matter state, (solid, liquid or gaseous) and its semi states, or can be implemented in any combination thereof.

In a further embodiment, this smart product has been designed and implemented to provide tailored quality of water as per regulatory standards and/or fitness requirement to stay healthy.

In a further embodiment, part or a complete module is used in smart cups, smart food or pharmaceutical drugs, preservation and its transportation of and so on.

### BRIEF DESCRIPTION OF THE DRAWINGS

Features and advantages of the example embodiments, and the manner in which the same are accomplished, will become apparent with reference to the following detailed description taken in conjunction with the accompanying drawings.

FIG. 1 is a diagram illustrating an apparatus for smart utilization and storage of resources in accordance with an example embodiment.

FIG. 2: is a diagram illustrating few modules of the invention “an apparatus for smart resource storage”.

FIG. 3: is a diagram illustrating few possible models of resource storage units.

FIG. 4: is a diagram illustrating artificial enabled learning modules of the resource storage apparatus.

These and other objects, features and advantages of the embodiments of the invention will become apparent from the following detailed description, which is to be read in connection with the accompanying drawings.

### DETAILED DESCRIPTION

Principles of the present invention include a systematic approach-based product of an apparatus for smart utilization and storage of resources proposed at appropriate locations.

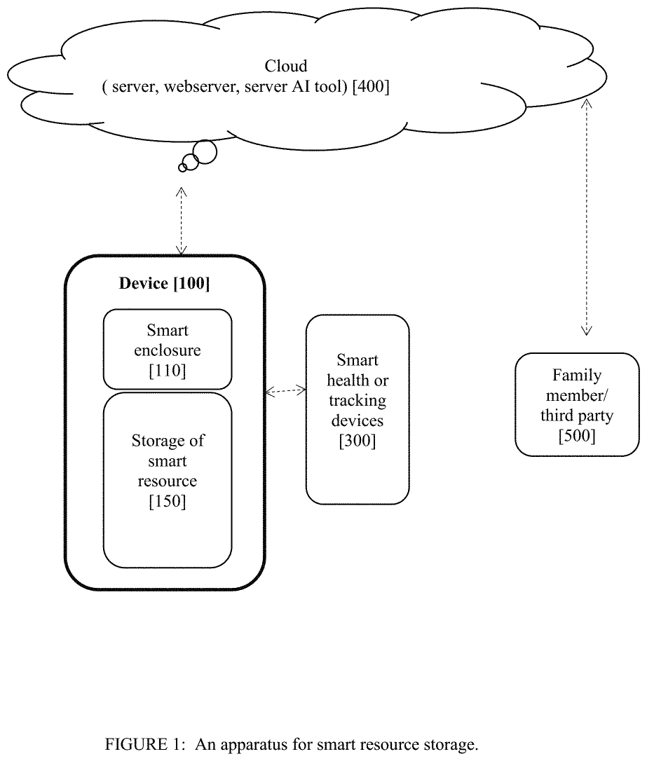

Reference made to FIG. 1, which illustrates an exemplary embodiment of an apparatus for diagnostic and prognostic-based identification, storage and purification of resources. The apparatus primarily consists of a Device 100, Smart enclosure 110, Storage of smart resource 150, which are connected to a Smart health or tracking devices 300, Family member/third party 500 and Cloud environment over possible connection, communication methods and technologies.

In a further embodiment, the resource may be available in any state of matter, say solid, liquid or gaseous or in its semi state.

In a further embodiment, the device consists of either two parts; one is the resource storing enclosure storage of smart resource 150, and the other is smart enclosure 110, or in a few cases both are embedded into single system 100.

In a further embodiment, the smart modules of the Device 100 is connected to Smart health or tracking devices 300 and in turn connected to Family member/consultant/medical provider or user 500, either through direct or through Cloud server 400 over possible communication technologies.

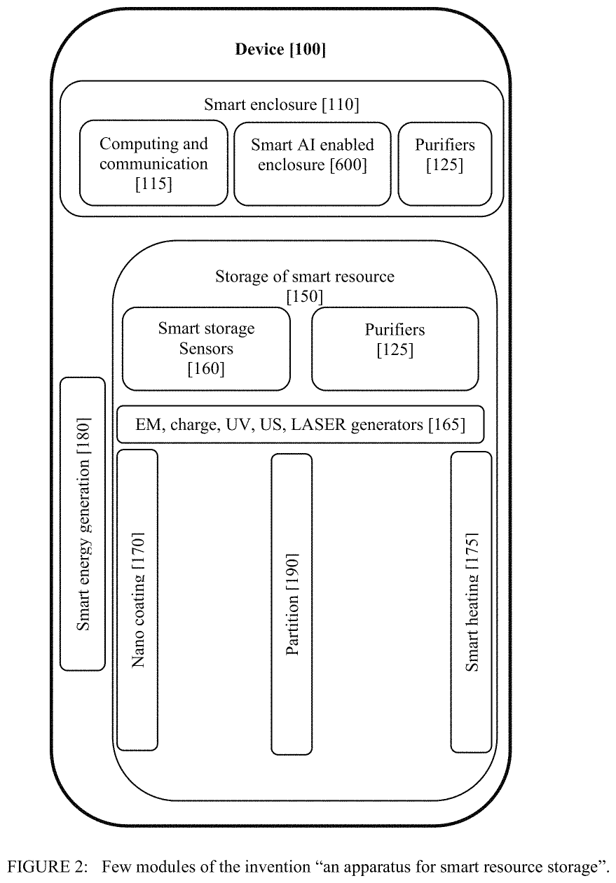

In a further embodiment, FIG. 2, illustrates few modules of the invention “an apparatus for smart resource storage”. Device 100 is having modularly connected modules such as smart enclosure 110, computing and communication 115, Smart AI enabled enclosure 600, Purifiers 125, Storage of smart resource 150, Smart storage Sensors 160, Purifiers 125, electromagnetic (EM), charge, ultra violet (UV), ultra sound (US), Light amplification by simulated emitting radiation (LASER) generators 165, Smart energy generation 180, Nano coating 170, Partition 190 and Smart heating 175.

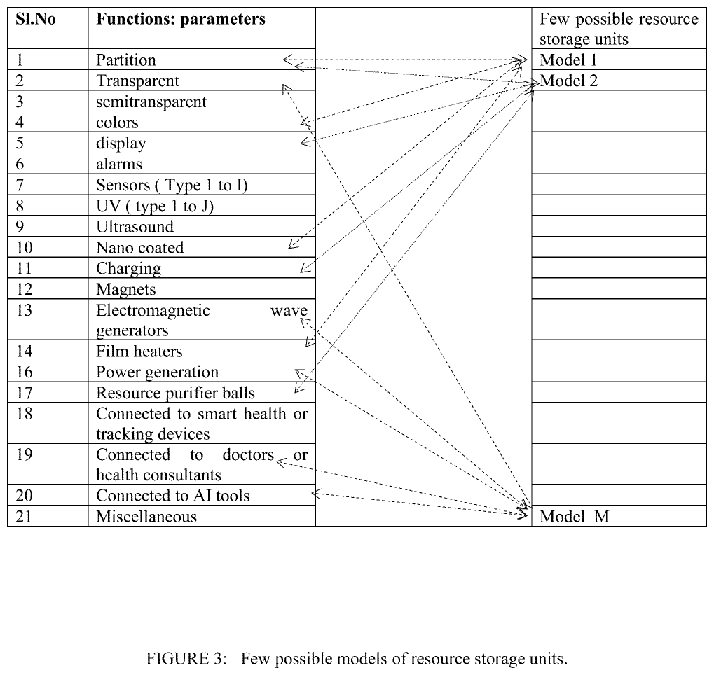

In a further embodiment, the FIG. 3: illustrates few possible models of resource storage units by customised requirements of the user. Each model is generated and manufactured with various distinct and different parameters and functions associated, namely Partition, Transparent or semi-transparent, colours, display, alarms, Sensors (Type 1 to I), UV (type 1 to J), Ultrasound, Nano coated material, Charging, magnets, Electromagnetic wave generators, Film heaters, Power generation, Resource purifier balls, Connected to smart health or tracking devices, Connected to doctors or health consultants, Connected to AI tools and so on.

In a further embodiment, user can design or choose several resource storage units based on his/her requirement by various permutation and combinations.
Model 1 of the resource storage having four features, namely Partition, colours, charging, resource purifier balls.
Model 2 of the resource identifier and storage has four features, namely Partition, display, charging, resource purifier balls.
Model M of the resource identifier and storage has six features, namely transparent, Electromagnetic wave generators, Power generation, connected to doctors or health consultants, Connected to AI tools

In a further embodiment, the resource storage unit and its enclosure are made of transparent, or semi-transparent or opaque with or without color shading material or bamboo or wood.

In a further embodiment, the apparatus made up of similar or dissimilar materials having single or multilayer partition with or without vacuum to hold the resource to maintain resources in cold and hot temperature condition.

In a further embodiment, the system modules would keep the resources unaffected by any external physical damages with leak and shock proof.

In a further embodiment, they keep the resources unaffected by any external chemical reactions or radiations or any combination thereof.

In a further embodiment, the resource can be in liquid form, water in particular.

In a further embodiment, the smart modules in the system would configure automatically the normal setting or override setting by users' requirement for water, drinking remainders, integrated with activity tracker, addition of mineral content, auto purification and other profile for the user and it would provide an alarm with display the status for the actions to be taken.

In a further embodiment, the smart device modules would track user's water intake, typically synchronize with a mobile phone app to keep user's hydration cues updated in real-time, and chart user's goals to drink pure water and stay healthy.

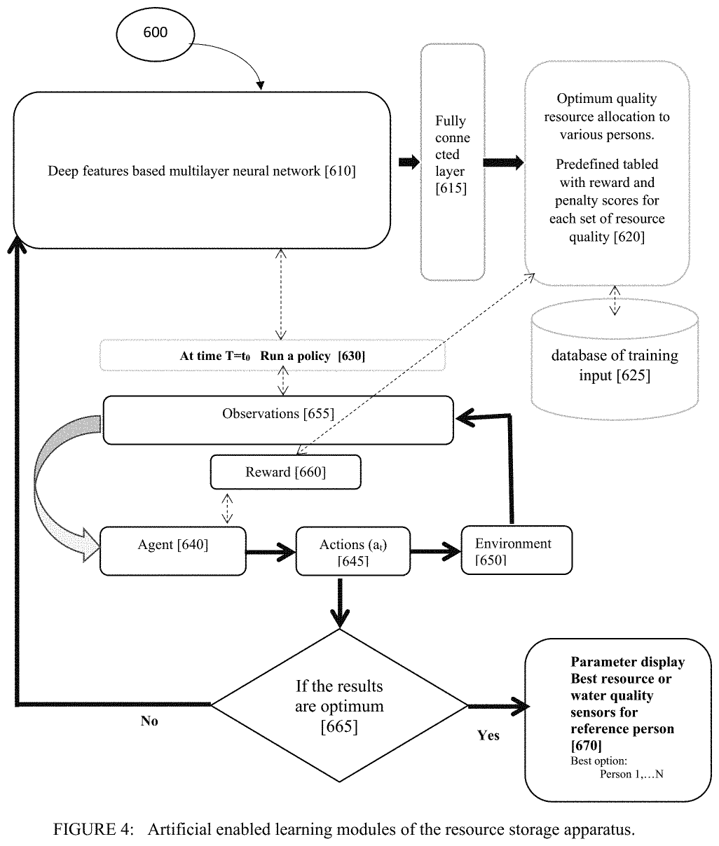

In a further embodiment, FIG. 4: describe one version implementation of a hybrid artificial enabled learning modules of the resource storage apparatus.

In a further embodiment, the multilayer deep neural network 610, made to train as per each user resource requirement profile with several associated parameters. This customised trained user profile is stored and used for validation in real time.

In a further embodiment, the output of deep neural networks fully connected layer 615, is connected to live database of training input 625. The training models can be prepared in the cloud or at the device level itself.

In a further embodiment, the optimum quality resource allocation to various users is computed and its stored in the predefined tabled with reward and penalty scores for each set of resource quality 620.

In a further embodiment, optimum result process of the parameter match is done through reinforcement leaning; the at time T=t0 Run a policy 630, with Agent 640, Actions (T=t0) 645, Environment 650, Observations 655, Reward 660, if the results are optimum 665. Later the smart system would displace the right information such as best resource or water quality sensors to be used for reference persons 670 requirement. Similarly learning module is optimised for other user requirements.

In a further embodiment, the several methods and techniques are used to purify the water in the resource or water storage unit, such as ultra violet light emitting diodes (say UV C LED) on periodic basis to neutralize the bacteria and pathogens.

In a further embodiment, the ultra sound methods and techniques used to improve the quality of water in the resource storage or bottle.

In a further embodiment, pumping more oxygen enhancing methods and techniques used to improve the quality of water in the resource storage or bottle.

In a further embodiment, several similar and dissimilar function-based sensors are used to reduce the toxic material and heavy metals available in the resource storage enclosure.

In a further embodiment, different type of water purifier such as Nano balls (organic and non-organic or plant extract) or water purifying Nano material coating inside the resource storage enclosure.

Advantages of the invention are that such a mechanism is easy to user and affordable in terms of cost and results in at least about 635 savings.

We claim:

1. An adaptively learning based apparatus having modularly intercoupled device for a utilization and storage of resources, the apparatus comprising:
a. at least one computing unit and device with communication modules;
b. at least one resource storage unit with or without an enclosure;
c. at least one smart health tracking devices;
d. at least one adaptively learning based deep learning enabled enclosure or lid;
e. at least one resource purifier;
f. at least one smart sensor in the storage and in the lid unit;
g. at least one purifier of matter available in any one of its state;
h. at least one electromagnetic wave, electric or magnetic charge/field, ultra violet, ultra sound, light amplification by stimulated emission of radiation (LASER) generator;
i. at least one smart energy generation;
j. at least one type of Nano material coating or balls;
k. at least one partition in the resource storage or enclosure or lid;
l. at least one smart heating module;
m. at least one model of the resource storage unit having various features and parameters; are connected to users or family members or others mobile device through direct or over the cloud server with all possible connecting and communication methods and techniques.

2. The apparatus claimed in claim 1, wherein the resource may be available in one or any form of state of the matter, namely solid, liquid or gas or in its semi states or in any combination thereof.

3. The apparatus claimed in claim 1, wherein the system has smart enclosure or lid and resource storing module, which embedded as single unit or having various detachable and refitting or closure mechanisms and methods.

4. The apparatus claimed in claim 3, wherein the resource storage unit and its enclosure is made of transparent, or semitransparent or opaque with or without color shading material or bamboo or wood or any combination thereof.

5. The apparatus claimed in claim 1, wherein the system in the apparatus made up of similar or dissimilar materials having single or multilayer partition with or without vacuum to hold the resource to maintain resources in cold and hot temperature condition with leak and shock proof.

6. The apparatus claimed in claim 5, wherein the system modules may available in any geometrical shape with or without folding or compressing features or in any combination thereof.

7. The apparatus claimed in claim 4, wherein the system modules would keep the resources unaffected by any external physical or chemical reactions or radiations or any combination thereof.

8. The apparatus claimed in claim 2, wherein one of the embodiments, resource can be in liquid form, water in particular.

9. The apparatus claimed in claim 1, wherein one or several modules in the modules such as smart enclosure, storage of smart resource and others fitted in single or multiple parts in the system based on selection of product modules of the apparatus.

10. The apparatus claimed in claim 1, wherein the smart modules in the system would configure automatically the normal setting or override setting by users' requirement for water, drinking reminders, integrated with activity tracker, addition of mineral content, auto purification and other profile for the user and it would provide an alarm with display the status for the actions to be taken.

11. The apparatus claimed in claim 10, where in the smart device modules would track user's water intake, typically synchronize with a mobile phone app to keep user's hydration cues updated in real-time, and chart user's goals to drink pure water and stay healthy.

12. The apparatus claimed in claim 1, wherein several methods and techniques are used to purify the water in the resource or water storage unit, such as ultra violet light emitting diodes (say UV C LED) on periodic basis to neutralize the bacteria and pathogens.

13. The apparatus claimed in claim 12, wherein profile based electric or magnetic charging mechanism added for the resource storage.

14. The apparatus claimed in claim 12, wherein ultra sound and oxygen enhancing methods and techniques used to improve the quality of water in the resource storage or bottle.

15. The apparatus claimed in claim 1, wherein several sensors used to detect toxic material and heavy metals and corresponding resources to reduce them are available in the resource storage enclosure.

16. The apparatus claimed in claim 1, wherein different type of water purifier such as Nano balls or water purifying Nano material coating inside the resource storage enclosure.

17. The apparatus claimed in claim 1, wherein the modules in the apparatus is made to work with internal rechargeable battery either through replacement or through auto charging mechanisms by thin film coated solar power generating films and associated learning-based charging circuit in the computing module of the apparatus.

18. The apparatus claimed in claim 1, where in the novel technique been implemented for water bottle and it can be used for any type of liquid or any of matter state, (solid, liquid or gaseous) and its semi states, or can be implemented in any combination thereof.

19. The apparatus claimed in claim 1, where in part or a complete module is used in smart cups, smart food or pharmaceutical drugs, preservation and its transportation of and so on.
